(19) United States
(12) Patent Application Publication
(10) Pub. No.: US 2023/0201103 A1
(43) Pub. Date: Jun. 29, 2023

(54) COSMETIC COMPOSITION COMPRISING AT LEAST 30% BY WEIGHT OF BRANCHED ALKANE AND A COMBINATION OF PARTICULAR SILICONES

(71) Applicant: L'OREAL, Paris (FR)

(72) Inventors: Pascale COTHIAS, Saint Ouen (FR); Carla BERGERE, Saint Ouen (FR); Léa CHABAS, Saint Ouen (FR)

(21) Appl. No.: 17/927,488

(22) PCT Filed: May 25, 2021
(86) PCT No.: PCT/EP2021/063878

§ 371 Date: Nov. 23, 2022

(30) Foreign Application Priority Data
May 26, 2020 (FR) 2005542

(51) Int. Cl. A61K 8/898; A61K 8/31; A61K 8/892; A61Q 5/12; A61K 8/37

(52) U.S. Cl. CPC A61K 8/898; A61K 8/31; A61K 8/892; A61Q 5/12; A61K 8/375

(57) ABSTRACT

One subject of the present invention is in particular an anhydrous cosmetic composition comprising: i) at least 30% by weight, relative to the total weight of the composition, of one or more branched alkanes having from 8 to 16 carbon atoms; ii) 5 to 15% by weight, relative to the total weight of the composition, of one or more dimethiconols; iii) one or more amino silicones; iv) one or more silicones different from the dimethiconols ii) and the amino silicones iii); and v) optionally one or more non-silicone fatty substances which have a melting point of less than or equal to 25° C. and are different from the branched alkanes i), the water content being of less than or equal to 4% by weight, relative to the total weight of the composition.

The present invention relates to a composition intended for the cosmetic treatment of keratin fibres, in particular of human keratin fibres such as the hair, comprising one or more branched alkanes, one or more dimethiconol(s), and also one or more amino silicones and one or more silicones different from dimethiconols and amino silicones.

The invention also relates to a process for the cosmetic treatment of keratin fibres, in which such a composition is applied to said fibres, and also to the use of this composition for caring for keratin fibres.

Hair is generally damaged and embrittled by the action of external atmospheric agents such as light and bad weather, and also by mechanical or chemical treatments, such as brushing, combing, dyeing, bleaching, permanent-waving and/or relaxing, or even repeated washing.

Hair is thus damaged by these various factors and may over time become dry, coarse, brittle or dull, in particular in fragile areas, and more particularly at the ends.

Thus, to overcome these drawbacks, it is common practice to resort to haircare treatments using compositions intended to condition the hair, giving it satisfactory cosmetic properties, in particular in terms of smoothness, sheen, softness, suppleness, lightness, a natural feel and good disentangling properties.

These haircare compositions, intended to be applied regularly to the hair, may be, for example, hair conditioners, masks or sera, and may be in the form of gels, hair lotions or care creams that are more or less thick.

It is in particular known practice to use compositions in the form of oily sera, containing combinations of silicone oils or non-silicone oils.

However, such compositions still too often result in excessive greasing of keratin fibres, which results in said fibres having a greasy or even tacky feel, and a lank, weighed-down visual appearance.

Furthermore, compositions containing silicone oils, once applied to the hair, frequently result in effects that are not very pleasant to the touch, in particular the hair has a tendency to be coarse and/or to squeak (that is to say produce an unpleasant sound), in particular when the strands of hair are rubbed together while sliding the fingers along the hair from the root to the end.

In addition, hair treated with such compositions is neither easy nor quick to dry.

Finally, the viscosity of these compositions is not always optimal, and does not make it possible to package them in a pump dispenser bottle.

The present invention aims to provide compositions which do not have the drawbacks mentioned above, and which are capable of conditioning keratin fibres in a long-lasting manner, in particular by giving them good cosmetic properties.

This aim is achieved by the present invention, a subject of which is in particular an anhydrous cosmetic composition, comprising:

i) at least 30% by weight, relative to the total weight of the composition, of one or more branched alkanes having from 8 to 16 carbon atoms;

ii) 5 to 15% by weight, relative to the total weight of the composition, of one or more dimethiconols;

iii) one or more amino silicones;

iv) one or more silicones different from the dimethiconols ii) and the amino silicones iii); and

v) optionally one or more non-silicone fatty substances which have a melting point of less than or equal to 25° C. and are different from the branched alkanes i), the water content being of less than or equal to 4% by weight, relative to the total weight of the composition.

The composition according to the invention makes it possible to condition keratin fibres very satisfactorily. In particular, it makes it possible to significantly improve the sheen, the softness, the ease of disentangling of the hair, without making it lank or making it greasy. The keratin fibres remain light, supple, and easy to style and to shape. They are neither visually greasy nor greasy to the touch, and do not squeak when touched.

This composition also makes it possible to nourish and repair damaged hair, in particular at the ends, and to facilitate the styling and straightening of curly or kinky hair.

Hair treated using this composition is easy and quick to dry, whether by heating by means for example of a hairdryer or naturally.

Finally, the composition according to the invention has a suitable viscosity for making it possible to package it in a pump dispenser bottle. It is neither too fluid nor too viscous, and can be dispensed by a pump system.

A subject of the invention is also a process for the cosmetic treatment of human keratin fibres such as the hair, comprising the application to said keratin fibres of a composition as defined according to the invention.

Another subject of the present invention relates to the use of the composition as defined above for conditioning keratin fibres, in particular human keratin fibres such as the hair.

Other subjects, characteristics, aspects and advantages of the invention will become even more clearly apparent on reading the description and the examples.

In the present description, and unless otherwise indicated:
the expression “at least one” is equivalent to the expression “one or more” and can be replaced therewith;
the expression “between” is equivalent to the expression “ranging from” and can be replaced therewith, and implies that the limits are included;
according to the present application, the term “keratin fibres” denotes human keratin fibres, and more particularly the hair.

The composition according to the invention is anhydrous, that is to say that it comprises a water content of less than or equal to 4% by weight, preferably less than or equal to 2% by weight, more preferentially less than or equal to 1% by weight, even more preferentially less than or equal to 0.5% by weight, relative to the total weight of the composition. Preferably, the composition according to the invention is totally free of water (0%).

Throughout the text hereinbelow, the term “silicone” denotes, in accordance with what is generally accepted, any organosilicon polymer or oligomer of linear or cyclic, branched or crosslinked structure, of variable molecular weight, obtained by polymerization and/or by polycondensation of suitably functionalized silanes, and constituted essentially of a repetition of main units in which the silicon atoms are linked together via oxygen atoms (siloxane bond —Si—O—Si—), optionally substituted hydrocarbon-based groups being directly bonded via a carbon atom to said silicon atoms. The hydrocarbon-based groups that are the most common are alkyl groups, in particular C₁-C₁₀ alkyl groups and in particular methyl, fluoroalkyl groups, the alkyl part of which is C₁-C₁₀, and aryl groups such as in particular phenyl groups.

In a manner known per se, the viscosity of the silicones, which is the kinematic viscosity, is measured at 25° C. and at atmospheric pressure (1 atm, 1.013×10⁵ Pa) in accordance with standard ASTM 445 Appendix C.

The weight-average molecular masses of the silicones may be measured by gel permeation chromatography (GPC) at ambient temperature (25° C.), as polystyrene equivalent. The columns used are μ styragel columns. The eluent is THF and the flow rate is 1 ml/minute. 200 μl of a 0.5% by weight solution of silicone in THF are injected. Detection is performed by refractometry and UV-metry.

Branched Alkanes i)

The composition according to the present invention comprises at least 30% by weight, relative to the total weight of the composition, of one or more branched alkanes i) having from 8 to 16 carbon atoms, preferably from 10 to 16 carbon atoms and more preferentially from 10 to 14 carbon atoms.

For the purposes of the invention, the term “alkane” is intended to mean a saturated linear or branched compound formed solely from carbon and hydrogen atoms.

The branched alkanes according to the invention correspond to the formula CₙH₂ₙ₊₂, with n being an integer ranging from 8 to 16, preferably from 10 to 16, more preferentially from 10 to 14.

Particularly preferably, the branched alkane i) is chosen from branched alkanes (or isoparaffins) comprising from 10 to 14 carbon atoms, in particular from C₁₁, C₁₂ and C₁₃ branched alkanes, alone or in a mixture; very particularly from isododecane, and mixtures of C₁₁-C₁₃ alkanes (INCI name: C₁₁-C₁₃ isoparaffin), and also mixtures of these compounds.

Advantageously, the branched alkane(s) i) are present in a total content of greater than or equal to 40% by weight, preferably greater than or equal to 50% by weight, more preferentially greater than or equal to 55% by weight, even more preferentially greater than or equal to 60% by weight, better still greater than or equal to 65% by weight, and even better still greater than or equal to 70% by weight, relative to the total weight of the composition.

According to a preferred embodiment, the branched alkane(s) i) are present in a total content ranging from 40% to 95% by weight, preferably from 50% to 90%, and more preferentially from 60% to 85% by weight, relative to the total weight of the composition.

Better still, the composition according to the invention may comprise one or more branched alkanes chosen from C₁₁, C₁₂ and C₁₃ branched alkanes, alone or in a mixture, in a total content of greater than or equal to 40% by weight, preferably greater than or equal to 50% by weight, more preferentially greater than or equal to 55% by weight, even more preferentially greater than or equal to 60% by weight, better still greater than or equal to 65% by weight, and even better still greater than or equal to 70% by weight, relative to the total weight of the composition.

According to a preferred embodiment, the composition according to the invention comprises one or more branched alkanes chosen from C₁₁, C₁₂ and C₁₃ branched alkanes, alone or in a mixture, in a total content ranging from 40% to 95% by weight, preferably from 50% to 90% by weight, and more preferentially from 60% to 85% by weight, relative to the total weight of the composition.

According to a particularly preferred embodiment, the composition comprises isododecane in a content of greater than or equal to 30% by weight, preferably greater than or equal to 35% by weight, and better still greater than or equal to 40% by weight, relative to the total weight of the composition.

Preferentially, the composition comprises isododecane and one or more branched alkanes different from isododecane chosen from among C₁₁ to C₁₃ branched alkanes; for example 30% to 60% by weight of isododecane, in particular 35% to 55% by weight of isododecane, relative to the total weight of the composition.

Dimethiconols ii)

The composition according to the invention comprises one or more dimethiconols ii).

The name dimethiconol denotes, in a manner known per se, polydimethylsiloxanes comprising dimethylsilanol end groups.

Preferably, the dimethiconol(s) according to the invention has (have) a weight-average molecular weight (Mw) of greater than 1000 daltons, preferably greater than 10000 daltons, more preferentially greater than 50000 daltons, better still greater than 100000 daltons.

By way of known compounds, mention may in particular be made of the compounds having the INCI name dimethiconol, such as those offered by the company Dow Corning under the trade names Dow Corning 1515 Gum, Dowsil 1515 Gum or Dowsil PMX 1505 Fluid.

The dimethiconol(s) ii) are present in a total content ranging from 5% to 15% by weight, and preferentially ranging from 5% to 10% by weight, relative to the total weight of the composition.

Amino Silicones iii)

The composition according to the invention also comprises one or more amino silicones iii). Said amino silicones iii) are different from the dimethiconols ii) above.

The term “amino silicone” denotes any silicone comprising at least one primary, secondary or tertiary amine or a quaternary ammonium group.

The weight-average molecular masses of these amino silicones may be measured by gel permeation chromatography (GPC) at ambient temperature (25° C.), as polystyrene equivalent. The columns used are μ steerage columns. The eluent is THF and the flow rate is 1 ml/minute. 200 μl of a 0.5% by weight solution of silicone in THF are injected. Detection is performed by refractometry and UV-metry.

Suitable amino silicones iii) which may be used in accordance with the present invention include, without being limited thereto, volatile and non-volatile, cyclic, linear and branched amino silicones having a viscosity ranging from 5×10⁻⁶ to 2.5 m²/s at 25° C., for example from 1×10⁻⁵ to 1 m²/s.

Preferably the amino silicone(s) iii) are chosen from:

a) the polysiloxanes corresponding to formula (IV):

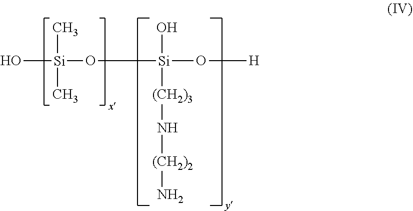

in which x′ and y′ are integers such that the weight-average molecular mass (Mw) is between 5000 and 500000 g/mol;

b) the amino silicones corresponding to formula (V):

R′ₐG₃₋ₐ-Si(OSiG₂)ₙ-(OSiG_{b}R′_{2-b})ₘ—O-SiG_{3-a′}-R′_{a′} (V)

in which:
G, which may be identical or different, denotes a hydrogen atom or a phenyl, OH, C₁-C₈ alkyl, for example methyl, or C₁-C₈ alkoxy, for example methoxy, group,
a and a′, which may be identical or different, denote 0 or an integer from 1 to 3, in particular 0, with the proviso that at least one from among a and a′ is equal to zero,
b denotes 0 or 1, in particular 1,
m and n are numbers such that the sum (n+m) ranges from 1 to 2000 and in particular from 50 to 150, n possibly denoting a number from 0 to 1999 and especially from 49 to 149, and m possibly denoting a number from 1 to 2000 and especially from 1 to 10;
R′, which may be identical or different, denotes a monovalent radical of formula —C_{q}H_{2q}L in which q is a number ranging from 2 to 8 and L is an optionally quaternized amino group chosen from the following groups:
—NR″-Q-N(R″)₂,
—N(R″)₂,
—N⁺(R″)₃A⁻,
—N⁺H(R″)₂A⁻,
—N⁺H₂(R″)A⁻,
—NR″-Q-N⁺(R″)H₂A⁻,
—NR″-Q-N⁺(R″)₂HA⁻ and
—NR″-Q-N⁺(R″)₃A⁻,

in which R″, which may be identical or different, denotes hydrogen, phenyl, benzyl, or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A⁻ represents a cosmetically acceptable anion, in particular a halide such as fluoride, chloride, bromide or iodide.

According to a first embodiment, the amino silicones corresponding to formula (V) are chosen from the silicones known as “trimethylsilylamodimethicone”, corresponding to formula (VI):

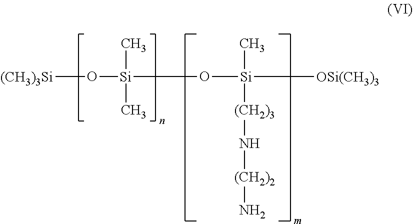

in which m and n are numbers such that the sum (n+m) ranges from 1 to 2000 and in particular from 50 to 150, n possibly denoting a number from 0 to 1999 and especially from 49 to 149, and m possibly denoting a number from 1 to 2000 and especially from 1 to 10.

According to a second embodiment, the amino silicones corresponding to formula (V) are chosen from the silicones of formula (VII) below:

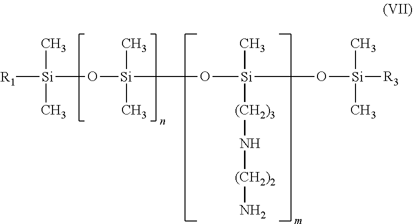

in which:
m and n are numbers such that the sum (n+m) ranges from 1 to 1000, in particular from 50 to 250 and more particularly from 100 to 200; n denoting a number from 0 to 999 and especially from 49 to 249 and more particularly from 125 to 175, and m denoting a number from 1 to 1000, especially from 1 to 10 and more particularly from 1 to 5; and
R₁, R₂ and R₃, which may be identical or different, represent a hydroxyl or C₁-C₄ alkoxy radical, at least one of the radicals R₁ to R₃ denoting an alkoxy radical.

Preferably, the alkoxy radical is a methoxy radical.

The hydroxy/alkoxy mole ratio preferably ranges from 0.2:1 to 0.4:1 and preferably from 0.25:1 to 0.35:1 and more particularly is equal to 0.3:1.

The weight-average molecular mass (Mw) of these silicones preferably ranges from 2000 to 1000000 g/mol and more particularly from 3500 to 200000 g/mol.

According to a third embodiment, the amino silicones corresponding to formula (V) are chosen from the silicones of formula (VIII) below:

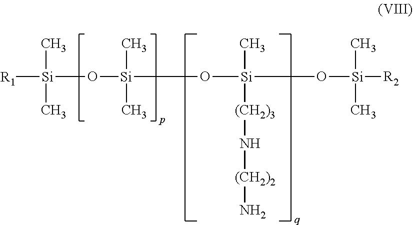

in which:
p and q are numbers such that the sum (p+q) ranges from 1 to 1000, in particular from 50 to 350 and more particularly from 150 to 250; p denoting a number from 0 to 999 and especially from 49 to 349 and more particularly from 159 to 239, and q denoting a number from 1 to 1000, especially from 1 to 10 and more particularly from 1 to 5; and
R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxy/alkoxy mole ratio generally ranges from 1:0.8 to 1:1.1 and preferably from 1:0.9 to 1:1 and more particularly is equal to 1:0.95.

The weight-average molecular mass (Mw) of the silicone preferably ranges from 2000 to 200 000 g/mol, more preferentially from 5000 to 100 000 g/mol and in particular from 10 000 to 50 000 g/mol.

The commercial products comprising silicones of structure (VII) or (VIII) may include in their composition one or more other amino silicones, the structure of which is different from formula (VII) or (VIII).

A product containing amino silicones of structure (VII) is sold by the company Wacker under the name Belsil® ADM 652.

A product containing amino silicones of structure (VIII) is sold by Wacker under the name Fluid WR 1300®. Another product containing amino silicones of structure (VIII) is sold by Wacker under the name Belsil ADM LOG 1®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-mean size of the silicone particles in the emulsion generally ranges from 3 nm to 500 nanometres. Preferably, in particular as amino silicones of formula (VIII), use is made of microemulsions of which the mean particle size ranges from 5 nm to 60 nm (limits included) and more particularly from 10 nm to 50 nm (limits included). Thus, use may be made according to the invention of the microemulsions of amino silicone of formula (VIII) sold under the names Finish CT 96 E® or SLM 28020® by the company Wacker.

According to a fourth embodiment, the amino silicones corresponding to formula (V) are chosen from the silicones of formula (IX) below:

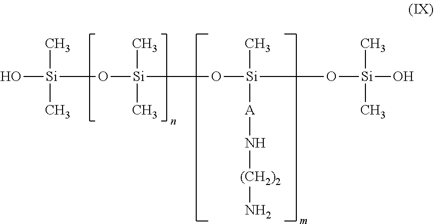

in which:
m and n are numbers such that the sum (n+m) ranges from 1 to 2000 and in particular from 50 to 150, n denoting a number from 0 to 1999 and especially from 49 to 149, and m denoting a number from 1 to 2000 and especially from 1 to 10; and
A denotes a linear or branched alkylene radical having from 4 to 8 carbon atoms and preferably 4 carbon atoms. This radical is preferably linear.

The weight-average molecular mass (Mw) of these amino silicones preferably ranges from 2000 to 1000000 g/mol and more particularly from 3500 to 200 000 g/mol.

A silicone corresponding to this formula is, for example, Xiameter MEM 8299 Emulsion from Dow Corning.

According to a fifth embodiment, the amino silicones corresponding to formula (V) are chosen from the silicones of formula (X) below:

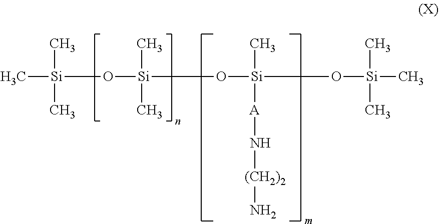

in which:
m and n are numbers such that the sum (n+m) ranges from 1 to 2000 and in particular from 50 to 150, n possibly denoting a number from 0 to 1999 and especially from 49 to 149, and m possibly denoting a number from 1 to 2000 and especially from 1 to 10; and
A denotes a linear or branched alkylene radical having from 4 to 8 carbon atoms and preferably 4 carbon atoms. This radical is preferably branched.

The weight-average molecular mass (Mw) of these amino silicones preferably ranges from 500 to 1000000 g/mol and more particularly from 1000 to 200 000 g/mol.

A silicone corresponding to this formula is, for example, DC2-8566 Amino Fluid from Dow Corning.

c) the amino silicones corresponding to formula (XI):

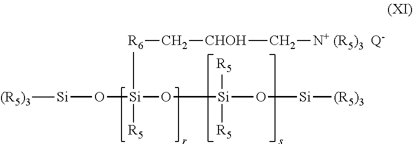

in which:
R₅ represents a monovalent hydrocarbon-based radical having from 1 to 18 carbon atoms, and in particular a C₁-C₁₈ alkyl or C₂-C₁₈ alkenyl, for example methyl, radical;
R₆ represents a divalent hydrocarbon-based radical, in particular a C₁-C₁₈ alkylene radical or a divalent C₁-C₁₈, for example C₁-C₁₈, alkyleneoxy radical linked to the Si via an SiC bond;
Q⁻ is an anion such as a halide ion, in particular chloride, or an organic acid salt, in particular acetate;
r represents a mean statistical value ranging from 2 to 20 and in particular from 2 to 8;
s represents a mean statistical value ranging from 20 to 200 and in particular from 20 to 50;

d) the quaternary ammonium silicones of formula (XII):

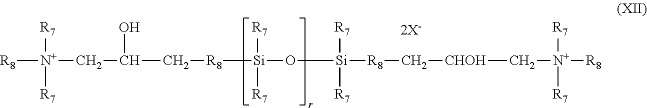

in which:
R₇, which may be identical or different, represent a monovalent hydrocarbon-based radical having from 1 to 18 carbon atoms, and in particular a C₁-C₁₈ alkyl radical, a C₂-C₁₈ alkenyl radical or a ring comprising 5 or 6 carbon atoms, for example methyl;
R₆ represents a divalent hydrocarbon-based radical, in particular a C₁-C₁₈ alkylene radical or a divalent C₁-C₁₈, for example C₁-C₁₈, alkyleneoxy radical linked to the Si via an SiC bond;
R₈, which may be identical or different, represent a hydrogen atom, a monovalent hydrocarbon-based radical having from 1 to 18 carbon atoms, and in particular a C₁-C₁₈ alkyl radical, a C₂-C₁₈ alkenyl radical or a radical —R₆—NHCOR₇;
X⁻ is an anion such as a halide ion, in particular chloride, or an organic acid salt, in particular acetate;
r represents a mean statistical value ranging from 2 to 200 and in particular from 5 to 100.

Such amino silicones are in particular described in patent application EP-A 0 530 974.

e) the amino silicones of formula (XIII):

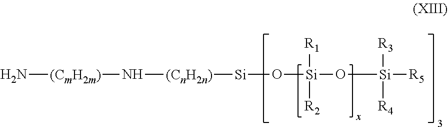

in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq/g;

f) multiblock polyoxyalkylenated amino silicones, of the type (AB)ₙ, A being a polysiloxane block and B being a polyoxyalkylenated block comprising at least one amine group.

Said silicones are preferably constituted of repeating units having the following general formulae:

[—(SiMe₂O)ₓSiMe₂-R—N(R″)—R′—O(C₂H₄O)ₐ(C₃H₆O)_{b}—R′—N(H)—R—]

or alternatively

[—(SiMe₂O)ₓSiMe₂-R—N(R″)—R′—O(C₂H₄O)ₐ(C₃H₆O)_{b}—]

in which:
a is an integer greater than or equal to 1, preferably ranging from 5 to 200 and more particularly ranging from 10 to 100;
b is an integer between 0 and 200, preferably ranging from 4 to 100 and more particularly between 5 and 30;
x is an integer ranging from 1 to 10 000 and more particularly from 10 to 5000;
R″ is a hydrogen atom or a methyl;
R, which may be identical or different, represent a linear or branched divalent C₂-C₁₂ hydrocarbon-based radical, optionally comprising one or more heteroatoms such as oxygen; preferably, R, which may be identical or different, denote an ethylene radical, a linear or branched propylene radical, a linear or branched butylene radical or a CH₂CH₂CH₂OCH₂CH(OH)CH₂— radical; preferentially, R denote a CH₂CH₂CH₂OCH₂CH(OH)CH₂— radical; and
R′, which may be identical or different, represent a linear or branched divalent C₂-C₁₂ hydrocarbon-based radical, optionally comprising one or more heteroatoms such as oxygen; preferably, R′, which may be identical or different, denote an ethylene radical, a linear or branched propylene radical, a linear or branched butylene radical or a CH₂CH₂CH₂OCH₂CH(OH)CH₂— radical; preferentially, R′ denote —CH(CH₃)—CH₂—.

The siloxane blocks preferably represent between 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1000000 g/mol and more particularly between 10000 and 200000 g/mol.

Mention may in particular be made of the silicones sold under the name Silsoft A-843 or Silsoft A+ by Momentive.

g) the amino silicones of formulae (XIV) and (XV):

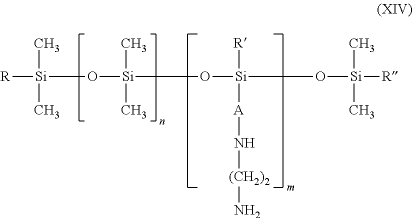

in which:
R, R′ and R′″, which may be identical or different, denote a C₁-C₄ alkyl group or a hydroxyl group,
A denotes a C₃ alkylene radical; and
m and n are numbers such that the weight-average molecular mass of the compound is between 5000 and 500000;

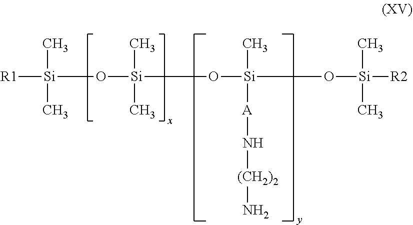

in which:
x and y are numbers ranging from 1 to 5000; preferably, x ranges from 10 to 2000 and more preferentially from 100 to 1000; preferably, y ranges from 1 to 100;
R₁ and R₂, which may be identical or different, preferably are identical, denote a linear or branched, saturated or unsaturated alkyl group comprising from 6 to 30 carbon atoms, preferably from 8 to 24 carbon atoms and more preferentially from 12 to 20 carbon atoms; and
A denotes a linear or branched alkylene radical having from 2 to 8 carbon atoms.

Preferably, A comprises from 3 to 6 carbon atoms, more preferentially 4 carbon atoms; preferably, A is branched.

Mention may be made in particular of the following divalent groups:

—CH₂CH₂CH₂— and —CH₂CH(CH₃)CH₂—.

Preferably, R₁ and R₂ are independent saturated linear alkyl groups comprising 6 to 30 carbon atoms, preferably 8 to 24 carbon atoms and in particular from 12 to 20 carbon atoms; mention may be made in particular of dodecyl, tetradecyl, pentadecyl, hexadecyl, heptadecyl, octadecyl, nonadecyl and eicosyl groups; and preferentially, R₁ and R₂, which may be identical or different, are chosen from hexadecyl (cetyl) and octadecyl (stearyl) groups.

The amino silicone(s) are preferably of formula (XV) with:
x ranging from 10 to 2000 and in particular from 100 to 1000;
y ranging from 1 to 100;
A comprising from 3 to 6 carbon atoms and in particular 4 carbon atoms;
preferably, A is branched; more particularly, A is chosen from the following divalent groups: —CH₂CH₂CH₂— and —CH₂CH(CH₃)CH₂—; and
R₁ and R₂ independently being saturated linear alkyl groups comprising from 6 to 30 carbon atoms, preferably from 8 to 24 carbon atoms and in particular from 12 to 20 carbon atoms; chosen in particular from dodecyl, tetradecyl, pentadecyl, hexadecyl, heptadecyl, octadecyl, nonadecyl and eicosyl groups; preferentially, R₁ and R₂, which may be identical or different, being chosen from hexadecyl (cetyl) and octadecyl (stearyl) groups.

A silicone of formula (XV) that is preferred is bis-cetearyl amodimethicone. Mention may be made in particular of the amino silicone sold under the name Silsoft AX by Momentive.

h) polysiloxanes and in particular polydimethylsiloxanes comprising primary amine groups at only one chain end or on side chains, such as those of formula (XVI), (XVII) or (XVIII):

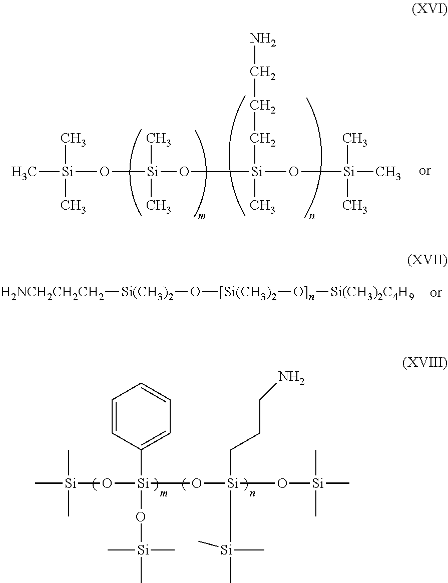

In formula (XVI), the values of n and m are such that the weight-average molecular mass of the amino silicone is between 1000 and 55000.

As examples of amino silicones of formula (XVI), mention may be made of the products sold under the names AMS-132, AMS-152, AMS-162, AMS-163, AMS-191 and AMS-1203 by the company Gelest and KF-8015 by the company Shin-Etsu.

In formula (XVII), the value of n is such that the weight-average molecular mass of the amino silicone is between 500 and 3000.

As examples of amino silicones of formula (XVII), mention may be made of the products sold under the names MCR-A11 and MCR-A12 by the company Gelest. In formula (XVIII), the values of n and m are such that the weight-average molecular mass of the amino silicone is between 500 and 50000.

As examples of amino silicones of formula (XVIII), mention may be made of the aminopropyl phenyl trimethicone sold under the name DC 2-2078 Fluid by the company Dow Corning.

i) and mixtures thereof.

Particularly preferably, the amino silicone iii) is an amodimethicone.

Advantageously, the amino silicone(s) iii) are present in a total content ranging from 0.5% to 10% by weight, preferably from 1% to 8% by weight, and more preferentially from 1.5% to 5% by weight, relative to the total weight of the composition.

According to a preferred embodiment, the composition according to the invention comprises one or more amodimethicones at a content of from 0.5% to 10% by weight, preferably from 1% to 8% by weight, and more preferentially from 1.5% to 5% by weight, relative to the total weight of the composition.

Advantageously, the weight ratio between the total content of the dimethiconol(s) ii) and the total content of the amino silicone(s) iii) is greater than or equal to 1, preferably greater than or equal to 2, more preferentially ranges from 2.5 to 5 and better still ranges from 3 to 4.

Silicones iv) Different from the Dimethiconols ii) and the Amino Silicones iii)

The composition according to the invention also comprises one or more silicones iv) different from the dimethiconols ii) and the amino silicones iii).

The silicone(s) iv) are different from the dimethiconols ii), that is to say that they are not polydimethylsiloxanes comprising dimethylsilanol end groups.

The silicone(s) iv) are different from the amino silicones iii), that is to say that they do not comprise any primary, secondary or tertiary amine or any quaternary ammonium group.

The silicones usable in the invention may be solid or liquid and volatile or non-volatile; they are preferably liquid and non-volatile.

For the purposes of the present invention, the term “volatile silicone” is understood to mean a silicone which is capable of evaporating on contact with the skin or the hair in less than one hour, at ambient temperature and atmospheric pressure. Such a silicone has in particular a saturation vapour pressure ranging from 0.13 to 40 000 Pa (10⁻³ to 300 mmHg), in particular ranging from 1.3 to 13000 Pa (0.01 to 100 mmHg), and more particularly ranging from 1.3 to 1300 Pa (0.01 to 10 mmHg).

For the purposes of the present invention, the term “non-volatile silicone” is understood to mean a silicone having a saturation vapour pressure of less than 0.13 Pa (0.01 mmHg).

The silicones that may be used may be soluble or insoluble in the composition according to the invention; they may be in the form of oil, wax, resin or gum; silicone oils are preferred.

Silicones are in particular described in detail in Walter Noll's Chemistry and Technology of Silicones (1968), Academic Press.

The volatile silicones may be chosen from those with a boiling point of between 60° C. and 260° C. (at atmospheric pressure) and more particularly from:

i) cyclic polydialkylsiloxanes comprising from 3 to 7 and preferably 4 to 5 silicon atoms, such as
octamethylcyclotetrasiloxane and decamethylcyclopentasiloxane.

Mention may be made of the products sold under the name Volatile Silicone 7207 by Union Carbide or Silbione 70045 V 2 by Rhodia, Volatile Silicone 7158 by Union Carbide or Silbione 70045 V 5 by Rhodia.
cyclocopolymers of the dimethylsiloxane/methylalkylsiloxane type having the chemical structure:

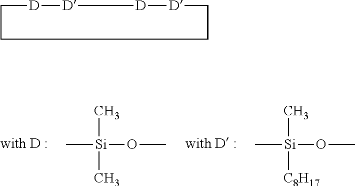

Mention may be made of Volatile Silicone FZ 3109 sold by the company Union Carbide;
mixtures of cyclic silicones with silicon-based organic compounds, such as the mixture of octamethylcyclotetrasiloxane and of tetratrimethylsilylpentaerythritol (50/50) and the mixture of octamethylcyclotetrasiloxane and of 1,1′-oxy(2,2,2′,2′,3,3′-hexatrimethylsilyloxy)bisneopentane;

ii) linear polydialkylsiloxanes having 2 to 9 silicon atoms, which generally have a viscosity of less than or equal to 5×10⁻⁶ m²/s at 25° C., such as decamethyltetrasiloxane.

Other silicones belonging to this category are described in the article published in Cosmetics and Toiletries, Vol. 91, January 76, pages 27-32, Todd & Byers Volatile Silicone Fluids for Cosmetics; mention may be made of the product sold under the name SH 200 by the company Toray Silicone.

Among the non-volatile silicones, mention may be made, alone or as a mixture, of polydialkylsiloxanes and in particular polydimethylsiloxanes (PDMS), polydiarylsiloxanes, polyalkylarylsiloxanes, silicone gums and resins, and also organopolysiloxanes (or organomodified polysiloxanes, or alternatively organomodified silicones) which are polysiloxanes including in their structure one or more organofunctional groups, generally attached via a hydrocarbon-based group, and preferably chosen from aryl groups, amine groups, alkoxy groups and polyoxyethylene or polyoxypropylene groups.

The organomodified silicones may be polydiarylsiloxanes, in particular polydiphenylsiloxanes, and polyalkylarylsiloxanes functionalized with the organofunctional groups mentioned previously. The polyalkylarylsiloxanes are particularly chosen from linear and/or branched polydimethyl/methylphenylsiloxanes and polydimethyl/diphenylsiloxanes.

Among the organomodified silicones, mention may be made of organopolysiloxanes comprising:
polyoxyethylene and/or polyoxypropylene groups optionally comprising C₆-C₂₄ alkyl groups, such as dimethicone copolyols, and in particular those sold by the company Dow Corning under the name DC 1248 or the oils Silwet® L 722, L 7500, L 77 and L 711 from the company Union Carbide; or alternatively (C₁₂)alkylmethicone copolyols, and in particular those sold by the company Dow Corning under the name Q2-5200;
thiol groups, such as the products sold under the names GP 72 A and GP 71 from Genesee;
alkoxylated groups, such as the product sold under the name Silicone Copolymer F-755 by SWS Silicones and Abil Wax® 2428, 2434 and 2440 by the company Goldschmidt;
hydroxylated groups, for instance polyorganosiloxanes bearing a hydroxyalkyl function;
acyloxyalkyl groups, such as the polyorganosiloxanes described in U.S. Pat. No. 4,957,732;
anionic groups of the carboxylic acid type, as described, for example, in EP 186 507, or of the alkylcarboxylic type, such as the product X-22-3701E from the company Shin-Etsu; or alternatively of the 2-hydroxyalkylsulfonate or 2-hydroxyalkylthiosulfate type, such as the products sold by the company Goldschmidt under the names Abil® S201 and Abil® S255;
hydroxyacylamino groups, such as the polyorganosiloxanes described in patent application EP 342 834; mention may be made, for example, of the product Q2-8413 from the company Dow Corning.

The silicones may also be chosen from polydialkylsiloxanes, among which mention may be made mainly of polydimethylsiloxanes bearing trimethylsilyl end groups. Among these polydialkylsiloxanes, mention may be made of the following commercial products:
the Silbione® oils of the 47 and 70 047 series or the Mirasil® oils sold by Rhodia, for instance the oil 70 047 V 500000;
the oils of the Mirasil® series sold by the company Rhodia;
the oils of the 200 series from the company Dow Corning, such as DC200 with a viscosity of 60000 mm²/s;
the Viscasil® oils from General Electric and certain oils of the SF series (SF 96, SF 18) from General Electric.

In this category of polydialkylsiloxanes, mention may also be made of the products sold under the names Abil Wax® 9800 and 9801 by the company Goldschmidt, which are poly(C₁-C₂₀)dialkylsiloxanes.

The polyalkylarylsiloxanes are chosen particularly from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, mention may be made of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Preferably, the silicone(s) iv) are chosen from polydimethylsiloxanes, more preferentially from among the linear (i.e. non-cyclic) polydimethylsiloxanes, better still the linear polydimethylsiloxanes which are liquid at 25° C., and better still from among the linear polydimethylsiloxanes which are liquid at 25° C. and non-volatile.

More preferentially, the silicone(s) iv) are chosen from the, preferably linear, polydimethylsiloxanes having a viscosity at 25° C. of less than or equal to 200×10⁻⁶ m²/s (200 cSt), more preferentially of less than or equal to 100×10⁻⁶ m²/s (100 cSt), better still of less than or equal to 50×10⁻⁶ m²/s (50 cSt), and even better still of less than or equal to 10×10⁻⁶ m²/s (10 cSt).

Advantageously, the total content of the silicone(s) iv) ranges from 1% to 20% by weight, preferably from 2% to 15% by weight, more preferentially from 4% to 10% by weight, relative to the total weight of the composition.

According to a preferred embodiment, the composition according to the invention comprises one or more polydimethylsiloxanes, in a total content ranging from 1% to 20% by weight, preferably from 2% to 15% by weight, and more preferentially from 4% to 10% by weight, relative to the total weight of the composition.

Preferably, the composition according to the invention is free of cyclomethicone, that is to say of cyclic polydimethylsiloxane, in particular of cyclomethicone comprising from 4 to 6 silicon atoms.

The expression “free of cyclomethicone” is understood to mean that the composition according to the invention does not comprise cyclomethicone, or that, when the composition according to the invention contains one or more cyclomethicones, the latter are present in a total content of less than or equal to 0.1% by weight, preferably of less than 0.05% by weight, relative to the total weight of the composition, even better still the composition is totally free of cyclomethicone (0% by weight).

Preferably, the composition according to the invention is free of cyclopentasiloxane(s).

The expression “free of cyclopentasiloxane(s)” is understood to mean that the composition according to the invention does not comprise cyclopentasiloxane(s), or that, when the composition according to the invention contains one or more cyclopentasiloxanes, the latter are present in a total content of less than or equal to 0.1% by weight, preferably of less than 0.05% by weight, relative to the total weight of the composition, even better still the composition is totally free of cyclopentasiloxane (0% by weight).

According to one particularly preferred embodiment, the composition according to the invention is free of decamethylcyclopentasiloxane (also referred to as D5).

The expression “free of decamethylcyclopentasiloxane” is understood to mean that the composition according to the invention does not comprise decamethylcyclopentasiloxane, or that, when the composition according to the invention contains decamethylcyclopentasiloxane, this compound is present in a total content of less than or equal to 0.1% by weight, preferably of less than 0.05% by weight, relative to the total weight of the composition, even better still the composition is totally free of decamethylcyclopentasiloxane (0% by weight).

Advantageously, the weight ratio between the total content of the silicone(s) iv) and the total content of the amino silicone(s) iii) is greater than or equal to 1, preferably greater than or equal to 2, more preferentially ranges from 2 to 4 and better still ranges from 2 to 3.

Advantageously, the weight ratio between the total content of the dimethiconol(s) ii) and the total content of the silicone(s) iv) is greater than or equal to 1, and preferably ranges from 1 to 2.

Advantageously, the weight ratio between the total content of the branched alkane(s) i) and the total content of the silicones (ii)+iii)+iv)) is greater than or equal to 1, preferably greater than or equal to 2, preferentially ranges from 3 to 6 and better still ranges from 4 to 5.

Non-silicone fatty substances v) which have a melting point of less than or equal to 25° C. and are different from the branched alkanes i)

The composition according to the invention may optionally also comprise one or more non-silicone fatty substances v) which have a melting point of less than or equal to 25° C., preferably of less than or equal to 20° C., at atmospheric pressure (1.013×10⁵ Pa), and are different from the branched alkanes i).

According to the invention, this or these fatty substance(s) are also referred to as “liquid fatty substance(s)” or “oil(s)”.

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name “MDSC 2920” by the company TA Instruments. In the present patent application, the melting points are determined at atmospheric pressure (1.013×10⁵ Pa).

The term “fatty substance” is understood to mean an organic compound that is insoluble in water at 25° C. and at atmospheric pressure (1.013×10⁵ Pa) (solubility of less than 5% by weight, and preferably less than 1% by weight, even more preferentially less than 0.1% by weight). They bear in their structure at least one hydrocarbon-based chain comprising at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

Advantageously, the liquid fatty substances that may be used in the present invention are not oxyalkylenated and do not contain any —COOH functions.

The term “non-silicone fatty substance” is understood to mean a fatty substance not containing Si—O bonds.

More particularly, the non-silicone fatty substance(s) v) according to the invention are chosen from non-silicone oils of animal origin, oils of triglyceride type of plant or synthetic origin, fluoro oils, liquid fatty alcohols, liquid fatty acid and/or fatty alcohol esters other than triglycerides, and mixtures thereof.

It is recalled that the fatty alcohols, esters and acids more particularly contain at least one saturated or unsaturated, linear or branched hydrocarbon-based group, comprising from 6 to 30 and better still from 8 to 30 carbon atoms, which is optionally substituted, in particular, with one or more hydroxyl groups (in particular 1 to 4). If they are unsaturated, these compounds can comprise one to three conjugated or non-conjugated carbon-carbon double bonds.

By way of hydrocarbon oils of animal origin, mention may be made of perhydrosqualene.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stéarinerie Dubois or those sold under the names Miglyol® 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethylperfluoromorpholine sold under the name PF 5052® by the company 3M.

The liquid fatty alcohols that are suitable for use in the invention are more particularly chosen from linear or branched, saturated or unsaturated alcohols, preferably unsaturated or branched alcohols, comprising from 6 to 30 carbon atoms, preferably from 8 to 30 carbon atoms. Mention may be made for example of octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol or linoleyl alcohol, and mixtures thereof.

As regards the liquid fatty acid and/or fatty alcohol esters other than the triglycerides mentioned above, mention may be made in particular of esters of saturated or unsaturated, linear C₁ to C₂₆ or branched C₃ to C₂₆ aliphatic monoacids or polyacids and of saturated or unsaturated, linear C₁ to C₂₆ or branched C₃ to C₂₆ aliphatic monoalcohols or polyalcohols, the total carbon number of the esters being greater than or equal to 6, more advantageously greater than or equal to 10.

Preferably, for the esters of monoalcohols, one at least of the alcohol or of the acid from which the esters of the invention result is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; ethyl palmitate, isopropyl palmitate, such as 2-ethylhexyl palmitate, 2-octyldecyl palmitate; alkyl myristates such as isopropyl 2-octyldodecyl myristate; isobutyl stearate; 2-hexyldecyl laurate, and mixtures thereof.

Preferably, among the monoesters of monoacids and of monoalcohols, use will be made of ethyl palmitate and isopropyl palmitate, alkyl myristates such as isopropyl myristate or ethyl myristate, isocetyl stearate, 2-ethylhexyl isononanoate, isodecyl neopentanoate and isostearyl neopentanoate, and mixtures thereof.

Still within the context of this variant, esters of C₄ to C₂₂ dicarboxylic or tricarboxylic acids and of C₁ to C₂₂ alcohols and esters of mono-, di- or tricarboxylic acids and of C₂ to C₂₆ di-, tri-, tetra- or pentahydroxy alcohols may also be used.

Mention may in particular be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; polyethylene glycol distearates, and mixtures thereof.

The composition may also comprise, as fatty ester, sugar esters and diesters of C₆ to C₃₀, preferably C₁₂ to C₂₂, fatty acids. It should be remembered that the term “sugar” is understood to mean oxygen-carrying hydrocarbon compounds which have several alcohol functions, with or without an aldehyde or ketone function, and which comprise at least 4 carbon atoms. These sugars can be monosaccharides, oligosaccharides or polysaccharides.

Mention may be made, as suitable sugars, for example, of sucrose, glucose, galactose, ribose, fucose, maltose, fructose, mannose, arabinose, xylose, lactose, and their derivatives, in particular alkylated derivatives, such as methylated derivatives, for example methylglucose.

The esters of sugars and of fatty acids may be chosen in particular from the group comprising the esters or mixtures of esters of sugars described previously and of linear or branched, saturated or unsaturated C₆ to C₃₀, preferably C₁₂ to C₂₂, fatty acids. If they are unsaturated, these compounds can comprise one to three conjugated or non-conjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates, arachidonates or mixtures thereof such as, in particular, the mixed oleo-palmitate, oleo-stearate and palmito-stearate esters.

More particularly, use is made of monoesters and diesters and in particular sucrose, glucose or methylglucose mono- or di-oleates, stearates, behenates, oleopalmitates, linoleates, linolenates and oleostearates, and mixtures thereof.

Mention may be made, by way of example, of the product sold under the name Glucate® DO by Amerchol, which is a methylglucose dioleate.

Preferably, use will be made of a liquid ester of a monoacid and of a monoalcohol.

Preferably, the fatty substance(s) v) are chosen from triglyceride oils of plant or synthetic origin, fatty esters having a melting point of less than or equal to 25° C. other than triglycerides, and mixtures thereof.

More preferentially, the fatty substance(s) v) are chosen from triglyceride oils of plant or synthetic origin and mixtures thereof.

Advantageously, the non-silicone fatty substance(s) v) are present in a content of greater than or equal to 5% by weight, relative to the total weight of the composition.

Advantageously, the weight ratio of the content of the branched alkane(s) i) and the content of the fatty substance(s) v) is greater than or equal to 1.

Colouring Agents

The composition according to the invention may optionally comprise one or more colouring agents, preferably chosen from direct dyes, pigments, and mixtures thereof.

The direct dye(s) may be chosen from ionic or nonionic direct dyes, preferably from nonionic dyes, anionic dyes, cationic dyes, and mixtures thereof, more preferentially from anionic dyes.

In a particular embodiment, the composition according to the invention also comprises one or more direct dyes, preferably one or more anionic direct dyes.

Additives

The cosmetic composition according to the present invention may also optionally comprise one or more additives, different from the compounds of the invention and among which mention may be made of cationic or anionic surfactants, and mixtures thereof, waxes, cationic, anionic, nonionic or amphoteric polymers, or mixtures thereof, antidandruff agents, anti-seborrhoea agents, vitamins and provitamins including panthenol, sunscreens, sequestrants, plasticizers, solubilizers, acidifying agents, mineral or organic thickeners, in particular polymeric thickeners, opacifiers, pearlescent agents, antioxidants, hydroxy acids, fragrances, preserving agents and fillers.

Of course, those skilled in the art will take care to choose this or these optional additional compounds so that the advantageous properties intrinsically associated with the composition according to the invention are not, or not substantially, detrimentally affected by the envisioned addition(s).

The above additives may generally be present in an amount, for each of them, of between 0 and 20% by weight relative to the total weight of the composition.

The composition according to the invention is advantageously clear, which gives it a particularly attractive aesthetic appearance that is highly sought after by users.

The composition according to the invention is advantageously in the form of a clear to translucent, more preferentially clear, fluid.

The clarity of the composition according to the invention can be characterized by the measurement of its turbidity, by turbidimetry (in NTU units). In the context of the present invention, the turbidity measurements were carried out with a turbidimeter, model 2100P from the company Hach.

Preferably, the turbidity of the compositions according to the invention, measured at ambient temperature (25° C.) and atmospheric pressure (1 atm), is less than 400 NTU units, better still less than 300 NTU units, in particular between 0 and 250 NTU units, or even between 0 and 200 NTU units, better still between 0 and 100 NTU units, even better still between 0.1 and 80 NTU units, or even between 0.2 and 50 NTU.

Preferably, the composition according to the invention has a dynamic viscosity at ambient pressure and temperature (25° C.; 1 atm) of less than 1000 mPa·s, in particular ranging from 80 to 500 mPa·s, or even from 90 to 400 mPa·s, and better still from 100 to 300 mPa·s.

The dynamic viscosity can be measured using an MCR 502 rotational rheometer from Anton Paar, with a cone/plate diameter 50 mm/1° (sanded steel at 5 μm) geometry; rotational speed 200 revolutions/min; measurements carried out at 25° C., 1 atm.

The composition according to the invention is advantageously packaged in a non-aerosol device, such as in particular a pump dispenser bottle.

A subject of the invention is also a process for the cosmetic treatment of human keratin fibres such as the hair, comprising the application to said keratin fibres of a composition as defined above.

The composition according to the invention may be applied to dry or wet keratin materials that have optionally been washed with a shampoo. Preferably, the composition according to the invention is applied to wet keratin fibres.

According to one embodiment, the keratin fibres are then rinsed with water, and optionally washed with a shampoo and then rinsed with water, before being dried or left to dry.

In this embodiment, the composition according to the invention is applied for a leave-on time that may range from 1 to 15 minutes and preferably from 2 to 10 minutes.

According to one preferred embodiment, the keratin fibres are not rinsed after application of the composition. They can then undergo heat and/or mechanical treatment for drying and/or shaping, for example straightening by means of a heating iron.

A subject of the invention is also the use of a composition as defined above for conditioning keratin fibres, in particular human keratin fibres such as the hair.

The composition can be used on wet or dry hair, in rinse-off or leave-on mode, and preferably in leave-on mode (that is to say that the keratin fibres are not rinsed after application of the composition).

The examples that follow serve to illustrate the invention without, however, being limiting in nature.

### EXAMPLES

In the examples that follow, all the amounts are indicated as weight percentages of active material relative to the total weight of the composition.

### Example 1

The following composition according to the invention was prepared from ingredients the active material contents (g AM) of which are indicated in the table below:

TABLE 1

| Composition | A (invention) |
|---|---|
| C₁₁-C₁₃ isoparaffins | 28 |
| Isododecane | q.s. 100 |
| Dimethiconol (1) | 7.875 |
| Dimethicone (2) | 6.8 |
| Caprylic/capric | 10 |
| triglycerides | |
| Amodimethicone (3) | 2.5 |
| Fragrance | 0.2 |

(1) DOWSIL PMX-1505 FLUID
(2) PDMS 6 cst
(3) DOWSIL AP-8568 AMINO FLUID

A haircare composition is obtained which can be used as an everyday hair oil.

After application, the composition dries very rapidly.

The hair does not appear visually greasy or dirty; it is smooth and non-greasy to the touch; it appears light and clean.

### Example 2

The following compositions B (according to the invention) and B′ (comparative) were prepared from ingredients the active material contents (g AM) of which are indicated in the table below:

TABLE 2

| Composition | B (invention) | B′ (comparative) |
|---|---|---|
| C₁₁-C₁₃ isoparaffins | 28 | — |
| Isododecane | 44.6 | 25 |
| Dimethiconol (1) | 7.9 | 7.9 |
| Dimethicone (2) | 6.6 | 54.2 |
| Caprylic/capric | 10 | 10 |
| triglycerides | | |
| Amodimethicone (3) | 2.5 | 2.5 |
| Fragrance | q.s. | q.s. |
| Argan oil | 0.1 | 0.1 |

(1) DOWSIL PMX-1505 FLUID
(2) PDMS 6 cst
(3) DOWSIL AP-8568 AMINO FLUID

Compositions B and B′ are then applied to the hair.

With composition B according to the invention, no grease is observed visually; the composition evaporates rapidly, the feel is very smooth, non-sticky and homogeneous.

Comparative composition B′ is more greasy in the hand, with the presence of clumps; grease is observed visually on the lock of hair, the composition does not evaporate and leaves a heavily coated, greasy feel, with transfer to the fingers.

Composition B according to the invention exhibits the following viscosity and turbidity values:
Viscosity=126 mPa·s (instrument: MCR502 Anton-Paar rheometer, cone/plate (50 mm) 1° sanded; protocol: flow curve at 38° C. as a function of shear speed from 0.1 to 1000 s⁻¹ over 26 points),
Turbidity=0.38 NTU (HACH model 2100 P turbidimeter)

### Example 3

The following compositions were prepared from ingredients the active material contents (g AM) of which are indicated in the table below:

TABLE 1

| Composition | C (invention) | C′ (comparative) |
|---|---|---|
| C₁₁-C₁₃ isoparaffins | 15.7 | 15.7 |
| Isododecane | q.s. 100 | q.s. 100 |
| Dimethiconol | 12 | 20 |
| Dimethicone | 9 | 9 |
| (viscosity: 5 CST) | | |
| Dimethicone (PMX-200 | 10 | 10 |
| SILICONE FLUID 2 CST) | | |
| Caprylic/capric | 10 | 10 |
| triglycerides | | |
| Amodimethicone | 2.5 | 2.5 |
| Fragrance | 0.3 | 0.3 |

Haircare compositions are obtained which can be used as an everyday hair oil.

The compositions are applied to the hair.

With composition C (according to the invention), the hair does not appear visually greasy or dirty; it is smooth and non-greasy to the touch; it appears light and clean.

1-18. (canceled)

19. A cosmetic composition comprising:
(i) one or more branched alkanes having from 8 to 16 carbon atoms;
(ii) one or more dimethiconols;
(iii) one or more amino silicones;
(iv) one or more silicones different from the dimethiconols (ii) and the amino silicones (iii); and
(v) optionally, one or more non-silicone fatty substances, different from the branched alkanes (i),
wherein the composition comprises water in an amount ranging from 0% to 4%,
wherein the total amount of branched alkanes (i) is at least 30%,
wherein the total amount of dimethiconols (ii) ranges from 5% to 15%,
wherein the melting point of one or more non-silicone fatty substances (v), if present, is less than or equal to 25° C., and
wherein all amounts are by weight, relative to the total weight of the composition.

20. The composition of claim 19, comprising one or more branched alkanes (i) chosen from branched alkanes having from 10 to 14 carbon atoms.

21. The composition of claim 19, wherein the total amount of branched alkanes (i) is at least 40% by weight, relative to the total weight of the composition.

22. The composition of claim 21, wherein the total amount of branched alkanes (i) ranges from 40% to 95% by weight, relative to the total weight of the composition.

23. The composition of claim 19, wherein the composition comprises isododecane in an amount of at least 30% by weight, relative to the total weight of the composition.

24. The composition of claim 19, wherein the total amount of dimethiconols (ii) ranges from 5% to 10% by weight, relative to the total weight of the composition.

25. The composition of claim 19, comprising one or more amino silicones (iii) chosen from:
a) polysiloxanes of formula (IV): 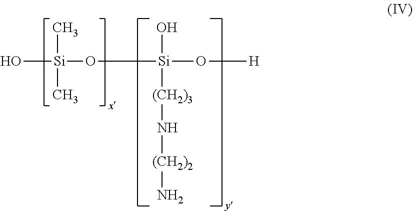
wherein x′ and y′ are independently chosen from integers such that a weight-average molecular mass (Mw) of the polysiloxane ranges from 5000 to 500,000 g/mol;
b) amino silicones of formula (V):
R′ₐG₃₋ₐ-Si(OSiG₂)ₙ-(OSiG_{b}R′_{2-b})ₘ—O-SiG_{3-a′}-R′_{a′} (V)
wherein:
G is independently chosen from a hydrogen atom, a phenyl group, an OH group, or a C₁-C₈ alkyl group;
a and a′ are independently chosen from 0 or an integer from 1 to 3, with the proviso that at least one a or a′ is equal to zero;
b is 0 or 1;
m and n are independently chosen from numbers such that the sum (n+m) ranges from 1 to 2000; and
R′ is independently chosen from a monovalent radical of formula —C_{q}H_{2q}L wherein q is a number ranging from 2 to 8 and L is an optionally quaternized amino group chosen from the following groups:
—NR″-Q-N(R″)₂,
—N(R″)₂,
—N⁺(R″)₃A⁻,
—N⁺H(R″)₂A⁻,
—N⁺H₂(R″)A⁻,
—NR″-Q-N⁺(R″)H₂A⁻,
—NR″-Q-N⁺(R″)₂HA⁻ and
—NR″-Q-N⁺(R″)₃A⁻,
wherein R″ is independently chosen from hydrogen, phenyl, benzyl, or a saturated monovalent hydrocarbon-based radical; Q is a linear or branched group of formula CᵣH₂ᵣ, r being an integer ranging from 2 to 6; and A⁻ represents a cosmetically acceptable anion;
c) amino silicones of formula (XI): 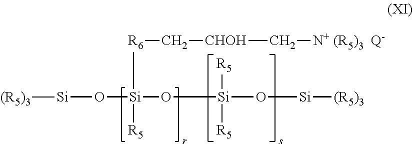
wherein:
R₅ represents a monovalent hydrocarbon-based radical having from 1 to 18 carbon atoms;
R₆ represents a divalent hydrocarbon-based radical;
Q⁻ is an anion;
r represents a mean statistical value ranging from 2 to 20; and
s represents a mean statistical value ranging from 20 to 200;
d) quaternary ammonium silicones of formula (XII): 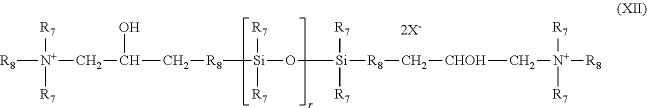
wherein:
R₇ is independently chosen from monovalent hydrocarbon-based radicals having from 1 to 18 carbon atoms;
R₆ represents a divalent hydrocarbon-based radical;
R₈ is independently chosen from a hydrogen atom, a monovalent hydrocarbon-based radical having from 1 to 18 carbon atoms, or a radical —R₆—NHCOR₇;
X⁻ is an anion; and
r represents a mean statistical value ranging from 2 to 200;
e) amino silicones of formula (XIII): 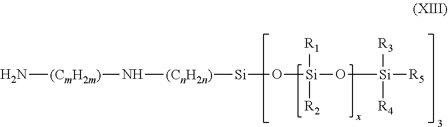
wherein:
R₁, R₂, R₃ and R₄ are independently chosen from a C₁-C₄ alkyl radical or a phenyl group;
R₅ denotes a C₁-C₄ alkyl radical or a hydroxyl group;
n is an integer ranging from 1 to 5;
m is an integer ranging from 1 to 5; and
x is chosen such that the amine number ranges from 0.01 to 1 meq/g;
f) multiblock polyoxyalkylenated amino silicones of the type (AB)ₙ, A being a polysiloxane block and B being a polyoxyalkylenated block comprising at least one amine group,
g) amino silicones of formulae (XIV) or (XV): 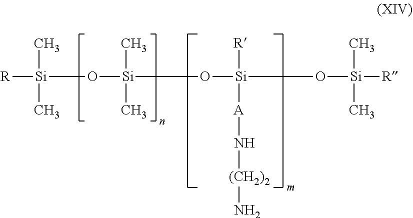
wherein:
R, R′ and R′″ are independently chosen from a C₁-C₄ alkyl group or a hydroxyl group;
A denotes a C₃ alkylene radical; and
m and n are independently chosen from numbers such that the weight-average molecular mass of the compound ranges from 5000 to 500,000; 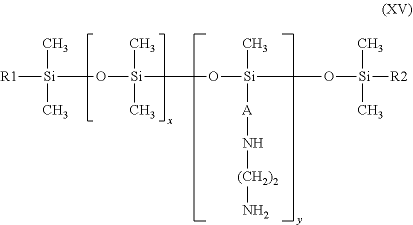
wherein:
x and y are independently chosen from numbers ranging from 1 to 5000;
R₁ and R₂ are independently chosen from linear or branched, saturated or unsaturated alkyl groups comprising from 6 to 30 carbon atoms; and
A denotes a linear or branched alkylene radical having from 2 to 8 carbon atoms,
h) polysiloxanes, or
i) mixtures thereof.

26. The composition of claim 19, wherein the total amount of amino silicones (iii) ranges from 0.5% to 10% by weight, relative to the total weight of the composition.

27. The composition of claim 19, comprising one or more silicones (iv) chosen from polydimethylsiloxanes.

28. The composition of claim 19, wherein the composition is free of decamethylcyclopentasiloxanes.

29. The composition of claim 19, wherein the weight ratio of the total amount of dimethiconols (ii) to the total amount of amino silicones (iii) is at least 1.

30. The composition of claim 19, wherein the weight ratio of the total amount of silicones (iv) to the total amount of amino silicones (iii) is at least 1.

31. The composition of claim 19, wherein the weight ratio of the total amount of dimethiconols (ii) to the total amount of silicones (iv) is at least 1.

32. The composition of claim 19, wherein the weight ratio of the total amount of branched alkanes (i) to the total amount of silicones ((ii)+(iii)+(iv)) is at least 1.

33. The composition of claim 19, comprising one or more non-silicone fatty substances (v) chosen from triglyceride oils of plant or synthetic origin, fatty esters having a melting point of less than or equal to 25° C. other than triglycerides, or mixtures thereof,
wherein the total amount of silicone fatty substances (v) is at least 5% by weight, relative to the total weight of the composition.

34. The composition of claim 19, further comprises one or more coloring agents.

35. A method for treating keratin fibers, comprising applying to the keratin fibers a composition comprising:
(i) one or more branched alkanes having from 8 to 16 carbon atoms;
(ii) one or more dimethiconols;
(iii) one or more amino silicones;
(iv) one or more silicones different from the dimethiconols (ii) and the amino silicones (iii); and
(v) optionally, one or more non-silicone fatty substances, different from the branched alkanes (i),
wherein the composition comprises water in an amount ranging from 0% to 4%,
wherein the total amount of branched alkanes (i) is at least 30%
wherein the total amount of dimethiconols (ii) ranges from 5% to 15%,
wherein the melting point of one or more non-silicone fatty substances (v), if present, is less than or equal to 25° C., and
wherein all amounts are by weight, relative to the total weight of the composition.

36. The method of claim 35, wherein the weight ratio of the total amount of dimethiconols (ii) to the total amount of amino silicones (iii) is at least 1.

37. The method of claim 35, wherein the weight ratio of the total amount of silicones (iv) to the total amount of amino silicones (iii) is at least 1.

38. A composition for treating keratin fibers comprising:
(i) one or more branched alkanes having from 8 to 16 carbon atoms;
(ii) one or more dimethiconols;
(iii) one or more amino silicones; and
(iv) one or more silicones different from the dimethiconols (ii) and the amino silicones (iii),
wherein the weight ratio of the total amount of dimethiconols (ii) to the total amount of silicones (iv) ranges from 1:1 to 2:1, and
wherein the composition comprises no more than 4% water.
